(19) United States
(12) Patent Application Publication
(10) Pub. No.: US 2006/0076506 A1
(43) Pub. Date: Apr. 13, 2006

(54) Micro-organism reduction in liquid by use of a metal halide ultraviolet lamp

(76) Inventor: Robert E. Duthie JR., East Aurora, NY (US)

Correspondence Address: HODGSON RUSS LLP, ONE M & T PLAZA, SUITE 2000, BUFFALO, NY, 14203-2391, US

(21) Appl. No.: 11/190,089

(22) Filed: Jul. 26, 2005

Related U.S. Application Data
(63) Continuation-in-part of application No. 09/903,825, filed on Jul. 11, 2001.

(51) Int. Cl. C02F 1/32

(52) U.S. Cl. 250/436; 422/24; 250/437

(57) ABSTRACT

A method and apparatus for disinfection/pasteurization of fluids. There is provided a mercury/gallium metal halide ultraviolet lamp enclosed within an ozone free metallic doped quartz envelope, an ozone free, metallic doped quartz enclosure for the lamp, an in-line stationary spiral or internal thread of single or multiple leads surrounding the enclosure, and a containment vessel having an inlet, an outlet and a chamber in fluid communication therewith defining a flow path for fluid to be disinfected/pasteurized. The lamp is operated at a wavelength range from about 100 nanometers to about 400 nanometers to introduce multiband ultraviolet radiation and minimal heat into the fluid with the enclosure preventing build up of ozone.

### CROSS REFERENCE TO A RELATED APPLICATION

This application is a continuation-in-part of co-pending application Ser. No. 09/903,825 filed Jul. 11, 2001 and entitled “Micro-Organism Reduction In Liquid By Use Of A Metal Halide Ultraviolet Lamp”, the disclosure of which is incorporated by reference.

### BACKGROUND OF THE INVENTION

This invention relates to the art of disinfection and pasteurization, and more particularly to a new and improved disinfection and pasteurization method and apparatus employing multiband ultraviolet light.

Since the detection of the micro-organism has increased within the food industry, non-thermal disinfection and pasteurization methods to reduce micro-organism contamination have also increased. Metal halide ultraviolet lamps have been employed in surface sterilization as described in U.S. Pat. No. 5,547,635 issued Aug. 20, 1996 and entitled “Sterilization Method and Apparatus,” the disclosure of which is hereby incorporated by reference.

### SUMMARY OF THE INVENTION

The present invention provides a method and apparatus employing non-thermal pasteurization utilizing technology involving surface sterilization with metal halide ultraviolet lamps. This unique non-thermal method of micro-organism reduction is achieved when a liquid is exposed to a high energy, metal halide, multiband ultraviolet lamp in an enclosed sealed chamber capable of allowing liquid flow into and out of a vessel. The radiation from the lamp will penetrate the liquid reducing the organism. The method comprises rapid heat transfer, titanium dioxide penetration, and multiband ultraviolet impregnation of the micro-organism within the liquid.

The following detailed description of the invention when read in conjunction with the accompanying drawings, is in such full, clear, concise and exact terms as to enable any person skilled in the art to which it pertains, or with which it is most nearly connected, to make and use the same.

### BRIEF DESCRIPTION OF THE DRAWING

FIG. 1 is a side elevational view of the apparatus according to the present invention;

FIG. 2 is an end elevational view of the apparatus of FIG. 1;

FIG. 3 is a perspective view of the apparatus of FIG. 1 with the outer housing removed;

FIG. 4 is a perspective view with parts removed of the apparatus of FIG. 1;

FIG. 5A is a fragmentary side elevational view of another form of the apparatus of the present invention;

FIG. 5B is a diagrammatic view further illustrating one of the units in the arrangement of FIG. 5B;

FIG. 6 is a graph providing definition of the ultraviolet light spectrum and the three wavelengths utilized in the present invention including germicidal ultraviolet light;

FIG. 7 is a spectral graph illustrating traditional germicidal ultraviolet light production;

FIG. 8 is a spectral graph illustrating multiband ultraviolet energy production utilized in the present invention;

FIG. 9 is a graph based on the data in Table 1;

FIG. 10 is a graph like that of FIG. 9 but giving additional information;

FIGS. 11A and 11B are diagrammatic views of a test set-up further illustrating the invention;

FIG. 12 is a graph illustrating results from comparisons using the set-up of FIGS. 11A and 11B;

FIGS. 13 and 14 are graphs presenting comparison data between a water purification system utilizing the invention and a water purification system utilizing conventional UV;

FIG. 15 is a schematic diagram illustrating particle path fluid dynamics in a flow processing apparatus;

FIG. 16 is a schematic diagram like FIG. 15 but for an alternative flow outlet arrangement;

FIG. 17 is a graph illustrating particle irradiation in the apparatus of FIG. 15;

FIG. 18 is a graph illustrating particle irradiation in the apparatus of FIG. 16;

FIG. 19 is a fluid particle path profile for the apparatus of the invention having the internal spiral geometry;

FIG. 20 is a graph illustrating fluid particle irradiation in the apparatus of the invention having the internal spiral geometry;

FIG. 21 is a diagrammatic view illustrating a pasteurizer incorporating the invention;

FIG. 22 is a schematic diagram illustrating particle path fluid dynamics in the pasteurizer of FIG. 21;

FIGS. 23A and 23B are graphs providing Reynolds Number plots associated with the illustration of FIG. 22;

FIG. 24 is a graph showing particle irradiation in the pasteurizer illustrated in FIGS. 21 and 22; and

FIGS. 25A and 25B are scanning electron microscope photographs further illustrating the invention.

### DETAILED DESCRIPTION OF THE INVENTION

The method and apparatus of the present invention employs non-thermal pasteurization utilizing research and technology involving surface sterilization with metal halide ultraviolet lamps. This unique non-thermal method of micro-organism reduction is achieved when a liquid is exposed to a high energy, metal halide, multiband ultraviolet lamp in an enclosed sealed chamber capable of allowing liquid flow into and out of a vessel. The radiation from the lamp will penetrate the liquid reducing the organism.

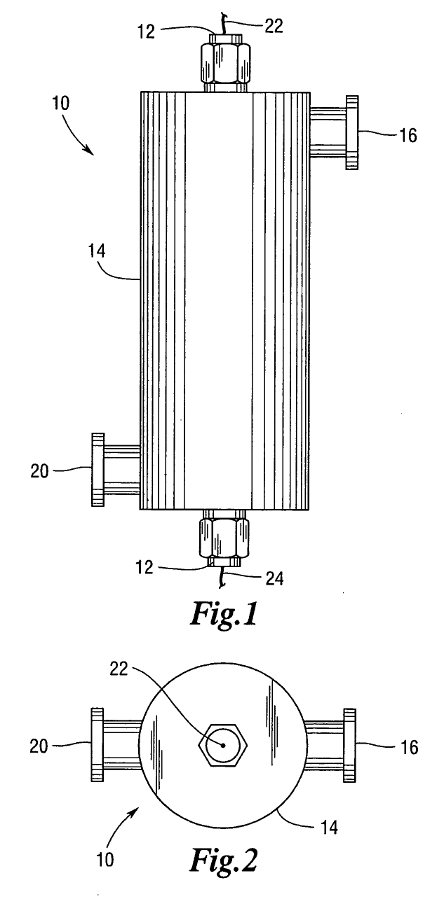

Referring to FIGS. 1-4, the lamp 10, with a metal halide configuration consisting of mercury and gallium, enclosed in an ozone free metallic doped quartz envelope, and having a wave length from about 100 to about 450 nanometers, is encapsulated in a second ozone free, metallic doped quartz tube 12. The metallic doping for the lamp envelope and tube 12 is titanium. Lamp 10 is similar to the lamp employed in the method and apparatus of the above-referenced U.S. Pat. No. 5,547,635. The quartz tube is sealed in a vessel 14 comprising an inlet 16, cylindrical chamber 18 enveloping the tube and an in-line stationary, spiral, internal thread of single lead 23 or multiple leads 23a, 23b surrounding the tube, and an outlet 20.

When the lamp 10 is ignited from an electronic ballast (not shown) in a circuit connected to the wires 22, 24 the lamp operates from about 100 nanometers to about 450 nanometers at a temperature ranging from about 400 degrees centigrade at the ends of the lamp to about 800 degrees centigrade at the center of the lamp. The afore-mentioned circuit can be similar to that shown in the above-referenced U.S. Pat. No. 5,547,635. The diameter of the vessel 14 is about twice the diameter of the tube 12 which in turn is twice the diameter of the lamp 10. The lamp 10 is approximately ½-inch diameter by about 16 inches in length for a ratio of about 100 watts per inch length at ½-inch diameter.

Liquid flow is dependent on the lamp output, spiral or threaded leads and pitch, and cylinder diameter. At constant velocity for a liquid, the internal spiral or thread increases the distance the liquid flows while in contact with the lamp and thereby increases the dwell time of the liquid in the lamp. Multiple threads increase mixing or turbulence of the liquid as it travels through the vessel. Flow rate for penetrations of a non-threaded vessel is about 0.1 gal/min./watt/vessel. This equates to a greater than five log reduction of micro-organism.

The process comprises rapid heat transfer and ultraviolet impregnation of the micro-organism within the liquid over the described period, and may also include titanium dioxide penetration. Multiband ultraviolet light and heat from lamp 10 are introduced to the liquid as the liquid follows along a flow path defined by the spiral through vessel 14. The quartz enclosure 12 allows ultraviolet light to be transmitted therethrough without appreciable buildup of ozone. The term rapid heat transfer used herein has the same general meaning as employed in describing the dry heat type of sterilization method. With rapid heat transfer, sterilization is time efficient with items drying quickly, in dry heat methods. In rapid heat transfer, as temperature increases, time decreases. It has been said that one way to get rapid heat transfer is to sterilize, not statically in a vessel but in a heat exchanger. The fluid is pumped continuously, and there is excellent energy economy by letting the hot sterilized medium exchange with the incoming medium. Applying the foregoing to the instant situation, the lamp 10, air and quartz tube 12 comprise the hot sterilized medium and the fluid or liquid flowing through vessel 14 comprises the exchange medium.

The unique non-thermal micro-organism reduction method additionally has implication in the medical industry.

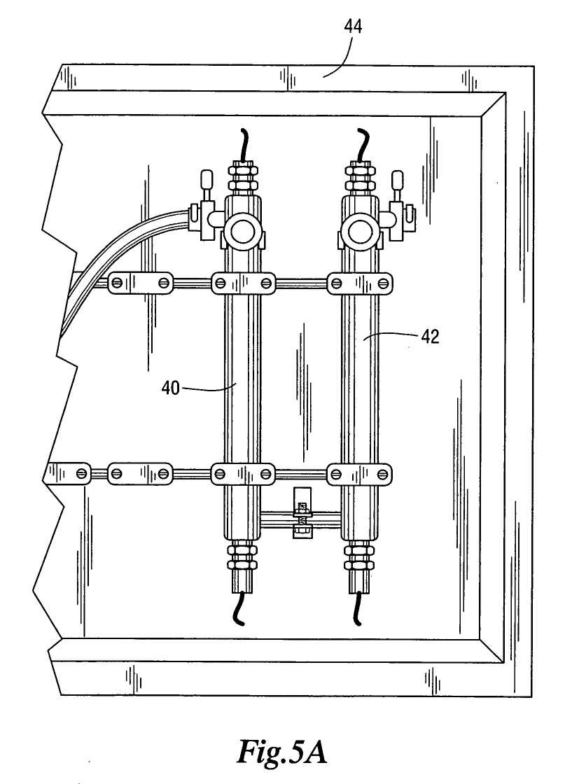

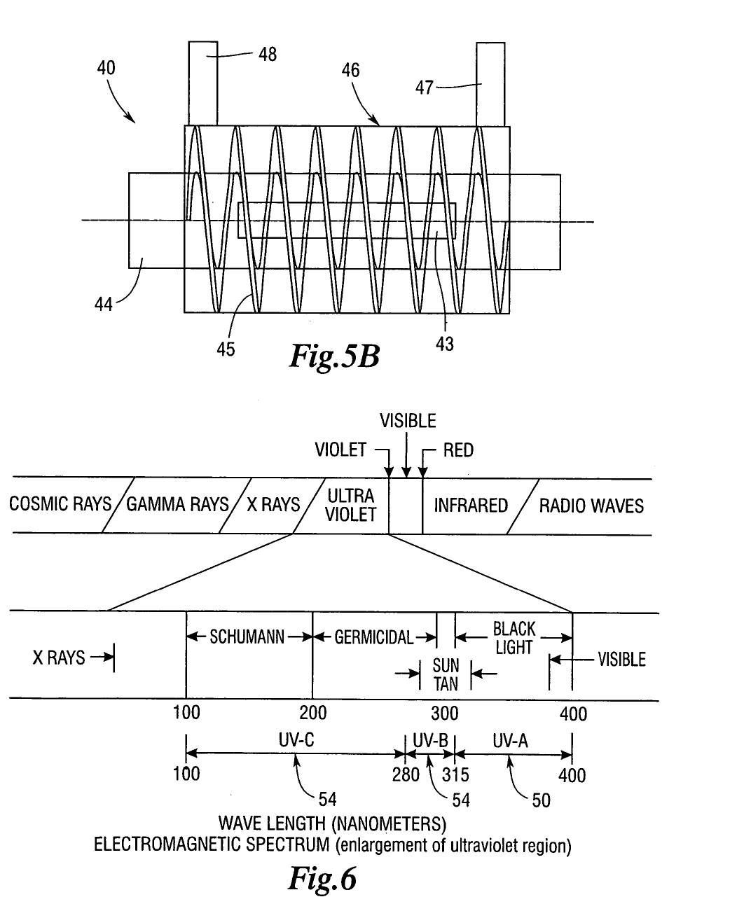

FIG. 5A illustrates another form of the apparatus of the invention wherein two units 40 and 42 are located within a housing 44 and connected in series. Each unit 40, 42 is similar to the apparatus shown in FIGS. 1-4 with an inlet on one end and an outlet on the other. FIG. 5B illustrates one of the units, for example unit 40, in more detail wherein a lamp 43, like lamp 10 of FIGS. 1-4, is within a quartz tube 44 surrounded by an auger 45 similar to threads 23 of FIGS. 1-4 which, in turn, is surrounded by vessel 46 having inlet 47 and outlet 48.

The method and apparatus of the present invention employs synergistic isogenous activated decontamination (a synergistic matrix of high energies simultaneously emitted from a single source) as a method of killing microorganisms by use of high intensity, broadband ultraviolet light combined with rapid heat transfer. This innovative and effective technology has particularly advantageous application to the food industry. However, the process has broad reaching potential applications far beyond the food industry, including the treatment of liquids, gasses, and solids.

The method and apparatus offer the following advantages over conventional methods of pasteurization or biological decontamination: processing speed, non-thermal, portable, nontoxic, no harmful radiation, electric and uncomplicated.

The method and apparatus of the present invention are further illustrated by the electromagnetic spectrum shown in FIG. 6. The specific wavelengths in the regions or bands identified UV-A, UV-B and UV-C which also are designated by the reference numerals 50, 52 and 54, show extremely high efficiency for producing micro-organism reduction or eradication when compared with the micro-organism reduction effects of current “state of the art” germicidal ultraviolet lamps operating only in the UV-C band designated 54 in FIG. 6. As shown in FIG. 6, band UV-A has a wavelength from about 315 nanometers to about 400 nanometers, band UV-B a wavelength from about 280 nanometers to about 315 nanometers and UV-C a wavelength from about 100 nanometers to about 280 nanometers.

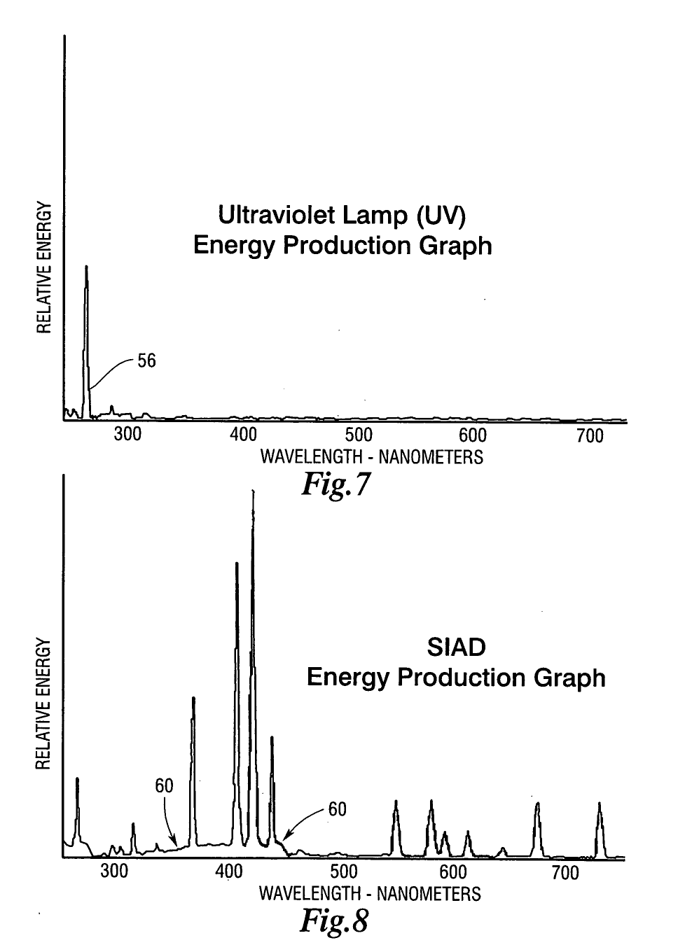

Current state of the art ultraviolet micro-organism reduction uses ultraviolet maps producing maximum energy only at 2,537 A or within the wavelength UV-C designated 54 in FIG. 6. In the spectral production graph of FIG. 7, the curve 56 illustrates the energy range of traditional, germicidal (UV-C) ultraviolet lamps such as these used in current water purification apparatus. The maximum bactericidal effectiveness of a germicidal (UV-C) ultraviolet lamp is manifested at 2,537 A, where 90% of exposed microorganisms are inactivated.

The mechanism of UV-C band germicidal action occurs as a result of the ultraviolet (UV-C) absorption at the 2,537A wavelength by the nucleic acids or their components. This is the initial event in the chain of reactions leading to demise. Most of the damage elicited by UV-C light results in the formation of cyclobutane-type dimers between adjacent thymines in deoxyribonucleic acid. Similar dimers also form in lesser amounts between cytosines and between thymine-cytosine pairs. The dimers are extremely stable and they block the normal replication and transcription of the DNA. These irreversible changes compromise cellular function, which eventually leads to death. The amount of energy necessary to destroy microorganisms depends primarily on the sensitivity of the organism. Thus, ultraviolet (UV-C) light causes adjacent thymines (or cytosines) in DNA to dimerize.

Laboratory studies indicate that 90% inactivation of most viruses and bacteria is possible by current UV-C germicidal lamps. Surviving microorganisms are left in a weakened state, interfering with replication and increasing their susceptibility to other inactivation methods, including heat and chemical agents.

Traditionally used UV-C light at 2,537A inactivates microorganisms by direct contact. Thus, microorganisms to be reduced would have to be directly exposed to the UV-C source. This could be termed “static sterilization and disinfection.” Sterilization is defined as the elimination or total destruction of microbial and viral life. Disinfection is the reduction of pathogenic microorganisms to a safe level by inhibiting cellular processes. Microorganisms may be shielded from direct UV-C by organic or inorganic matter. This protection from UV-C light is referred to as “screening and/or shadowing effect.” Screened microorganisms are not directly contacted by UV-C light. Therefore, screened microorganisms remain active following traditional UV-C irradiation.

Microorganism reduction using UV-C light is very limited and unreliable. However, the potential to sterilize does exist, as demonstrated by extensive research on airborne microbes. Additional reports support this claim, providing there is an unobstructed path of UV-C light to the target. For UV-C light to be considered a practical sterilization method, “shadow zones,” and “screening effects” must be eliminated.

Upon reviewing traditional germicidal UV-C ultraviolet microorganism reduction or “static sterilization and disinfection,” three important aspects of UV-C processing follow:
1. 90% activation of most microorganisms,
2. “screening and/or shadowing” affects the process, and
3. surviving microorganisms are left in a weakened state, interfering with replication and increasing their susceptibility to other inactivation methods, including heat and chemical agents.

The dilemma of traditional ultraviolet light (in the UV-C band) “static sterilization and disinfection” is overcome by means of a modified germicidal arc lamp providing simultaneous wavelength outputs of UV-A, UV-B and UV-C ultraviolet light previously described. In the spectral production graph of FIG. 8, curve 60 illustrates the energy range of the modified halide (UV-A, UV-B and UV-C) lamp used in the liquid purification apparatus of this invention. This improvement has generated the term “dynamic sterilization and disinfection.” Traditional germicidal, UV-C ultraviolet light sources lack the capacity to penetrate and cause molecular excitation by photon energy. The precise, simultaneous combination of UV-A, UV-B and UV-C ultraviolet light or “dynamic sterilization and disinfection” provides the capability of penetrating and causing molecular excitation. The excitation phenomenon involves the movement of organic and/or inorganic molecules, and the release of thermal energy.

Riboflavin (Vitamin B₂) occupies all cells including harmful microorganisms. Riboflavin will absorb UV-A, UN-B and UV-C light at 2200-2250A, 2660A, 3710A, 4440A, and 4750A. Absorption of ultraviolet at these wave lengths breaks apart the Riboflavin radical, destroying certain elements and leaving “free” radicals which cannot replicate. Dynamic sterilization and disinfection operates at the aforementioned wave lengths and thereby destroys the cell by disassembling the Riboflavin radicals. The “free” radicals formed from the disassembling of Riboflavin disrupt cellular metabolic activity and structure. In particular, this “free” radical operation interferes with the quality control function of protein in the cell, thereby rendering the cell susceptible to either self destruction or destruction by an external force or effect.

Riboflavin acts in living organisms as a coenzyme, flavin adenine dinucleotide (FAD). FAD is part of the mitochondrial electron transport chain and is the coenzyme for glutathione reductase (GR), an enzyme involved in the regeneration of glutahione. Glutathione is critical in reactivating Vitamin C. When Vitamin E is inactivated by neutralizing free radicals as those formed by the multiband ultraviolet light (UV-A, B, & C) disassembling Riboflavin, Vitamin C regenerates Vitamin E back to full activity. Vitamin E prevents oxidation of unsaturated fatty acids by trapping “free radicals.” This stabilizes and protects cell membranes and tissues sensitive to oxidation. Vitamin E has synergistic effects with Vitamin C, gluthathione, and other antioxidants. Thus, the disassembling of Riboflavin by multiband ultraviolet light (UV-A, B, & C) inactivates the rebuilding mechanism for the cell membrane, causing the cell membrane to be vulnerable to destruction from various energy sources such as heat and light. With the destruction of the cell membranes, the DNA chain, unprotected within the cellular structure, is exposed and vulnerable.

Utilizing a metal halide, multiband ultraviolet lamp with output ratios of about 35% UV-A, 40% UV-B and 25% UV-C according to the invention, the dynamic sterilization, and disinfection technology dramatically outperformed the traditional germicidal ultraviolet (UV-C) technology in microorganism reduction. The foregoing percentages apply to the distribution of the total relative energy (microwatts/cm²/sec or Joules/m²/sec) output from the lamp through the entire UV spectral range (UV-A, UV-B and UV-C). In particular, the relative energy distribution of the lamp according to the invention is approximately 35% MV/cm²/sec in UV-A (315-400 nm), 40% MW/cm²/sec in UV-B (280-315) and 25% MW/cm²/sec in UV-C (100-280 nm). The dynamic sterilization and disinfection lamps according to the invention operate in the broadband UV spectrum (1000A to 4000+A). These multiband lamps output high energy throughout the UV spectrum range. In addition, during operation the temperature at the center of the multiband lamp is in excess of 500C. Utilizing the multiband lamp, this unique synergism of traditional UV, high energy, broad band UV and heat transfer operate simultaneous on microorganisms. The total processing mechanism for dynamic sterilization and disinfection according to the present invention is termed synergistic isogenous activated decontamination (SIAD).

By way of example, a mercury/gallium lamp found to perform satisfactorily in the invention is commercially available from Voltarc Technologies Inc. of Fairfield, Conn. under part number 18522 UY15C/8FR/3654. The lamp glass is titanium doped silica quartz, and the lamp contains 20 Torr Argon gas, gallium in the amount of 1 Mg. and Mercury in the amount of 72 Mg. Data for an illustrative ⅜ inch diameter by 8-inch length version of the lamp is presented in Table 1, it being understood that other diameter and length versions of the lamp can be employed. In Table 1, the data is organized with the far left column being the starting wavelength in nanometers for the row, and each number in the row is the energy (microwats/sq.cm.x0.02 at 1 meter) for incremental wavelengths. For example, in the first row 2030 is the energy at 250 nanometers, 3807 the energy at 251 nanometers and proceeding to the right-hand end of the row, 759 is the energy at 259 nanometers. The far right column in Table 1 is the total energy for the preceding 10 wavelengths. Thus, 12410 is the sum of all ten entries in the first row. In Table 1, x represents energy and y represents wavelength.

TABLE 1

| | |
|---|---|
| MERCURY/GALLIUM METAL HALIDE LAMP | 1500 WATT |
| ⅜ inch Diameter × 8 inch Length | 500 ARC VOLT |
| Titanium Doped, Ozone-Free Quartz | X = 0.2561, Y = 0.2418 |
| MULTIPLY BY 0.02000000 FOR MICROWATTS/SQ.CM./NM., AT 1.000 METERS | |

| AVL | | | | | | | | | | | SUM |
|---|---|---|---|---|---|---|---|---|---|---|---|
| | | | | | | | | | | | /10 NM |
| 250 | 2030 | 3807 | 554 | 666 | 725 | 1265 | 780 | 1113 | 711 | 759 | 12410 |
| 260 | 754 | 369 | 236 | 493 | 734 | 1543 | 1241 | 604 | 311 | 332 | 6617 |
| 270 | 420 | 636 | 662 | 465 | 374 | 367 | 311 | 246 | 164 | 343 | 3990 |
| 280 | 512 | 480 | 323 | 194 | 174 | 270 | 652 | 1648 | 2248 | 1715 | 8215 |
| 290 | 989 | 603 | 581 | 1143 | 2590 | 3382 | 3229 | 2429 | 1363 | 823 | 17132 |
| 300 | 714 | 1194 | 1767 | 1316 | 527 | 327 | 273 | 228 | 226 | 214 | 6785 |
| 310 | 269 | 670 | 2402 | 3572 | 2094 | 589 | 284 | 221 | 194 | 184 | 10479 |
| 320 | 169 | 158 | 151 | 147 | 158 | 166 | 155 | 200 | 235 | 189 | 1728 |
| 330 | 143 | 144 | 164 | 372 | 654 | 476 | 186 | 171 | 198 | 190 | 2698 |
| 340 | 136 | 137 | 131 | 144 | 144 | 141 | 133 | 132 | 134 | 136 | 1368 |
| 350 | 133 | 131 | 129 | 132 | 134 | 134 | 139 | 155 | 168 | 161 | 1416 |
| 360 | 150 | 166 | 203 | 416 | 2817 | 6554 | 5492 | 2008 | 555 | 288 | 18647 |
| 370 | 241 | 224 | 231 | 230 | 235 | 220 | 195 | 178 | 166 | 181 | 2101 |
| 380 | 181 | 192 | 212 | 209 | 186 | 190 | 199 | 193 | 183 | 191 | 1936 |
| 390 | 218 | 224 | 194 | 187 | 183 | 181 | 191 | 203 | 207 | 226 | 2014 |
| 400 | 271 | 437 | 1926 | 7096 | 10849 | 8302 | 4311 | 2350 | 1415 | 713 | 37670 |
| 410 | 426 | 352 | 330 | 340 | 418 | 676 | 2985 | 9889 | 12554 | 8438 | 36410 |
| 420 | 5373 | 3270 | 1705 | 954 | 658 | 524 | 446 | 400 | 365 | 342 | 14038 |
| 430 | 335 | 336 | 325 | 400 | 766 | 3843 | 6504 | 3260 | 887 | 521 | 17177 |
| 440 | 437 | 417 | 413 | 414 | 388 | 324 | 253 | 196 | 161 | 139 | 3141 |
| | | | | | | | | | | | /20 NM |
| 450 | 125 | 106 | 98 | 97 | 101 | 109 | 112 | 105 | 91 | 81 | 2050 |
| 470 | 80 | 80 | 78 | 77 | 76 | 76 | 80 | 81 | 83 | 90 | 1602 |
| 490 | 106 | 115 | 111 | 110 | 115 | 117 | 110 | 85 | 65 | 56 | 1980 |
| 510 | 52 | 52 | 51 | 50 | 48 | 48 | 48 | 48 | 50 | 49 | 993 |
| 530 | 51 | 57 | 71 | 78 | 76 | 92 | 362 | 2232 | 3660 | 2048 | 17452 |
| 550 | 357 | 136 | 88 | 72 | 63 | 59 | 57 | 54 | 53 | 55 | 1988 |
| 570 | 66 | 116 | 599 | 1690 | 2131 | 1225 | 331 | 102 | 100 | 158 | 13036 |
| 590 | 144 | 79 | 53 | 50 | 49 | 48 | 47 | 48 | 48 | 53 | 1237 |
| 610 | 56 | 52 | 48 | 47 | 47 | 48 | 51 | 51 | 50 | 48 | 998 |
| 630 | 48 | 49 | 49 | 61 | 123 | 166 | 116 | 61 | 50 | 49 | 1544 |
| 650 | 48 | 48 | 49 | 48 | 49 | 49 | 48 | 51 | 54 | 177 | 1241 |
| 670 | 375 | 279 | 78 | 53 | 50 | 51 | 52 | 50 | 51 | 60 | 2202 |
| 690 | 81 | 74 | 56 | 52 | 52 | 50 | 53 | 53 | 56 | 61 | 1173 |
| 710 | 59 | 52 | 54 | 54 | 51 | 53 | 53 | 53 | 53 | 53 | 1070 |
| 730 | 53 | 51 | 51 | 53 | 54 | 54 | 54 | 54 | 56 | 55 | 1069 |
| 750 | 53 | 52 | 52 | 51 | 51 | 51 | 53 | 58 | 67 | 63 | 1104 |
| 770 | 66 | 69 | 65 | 57 | 59 | 62 | 57 | 55 | 57 | 45 | 1184 |
| 790 | 53 | 50 | 53 | 50 | 46 | 48 | | | | | 599 |

GRAND TOTAL = 2584.9
NIOSH Wt'd Spectral Irrad. (E, Eff., 250-315 nm) = 307.25
MPET (Max. permissible exposure time) = 9,7641 seconds

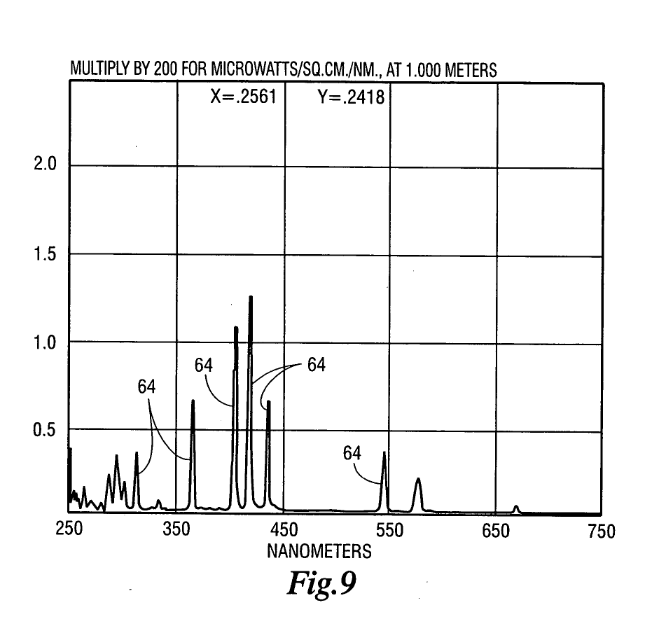

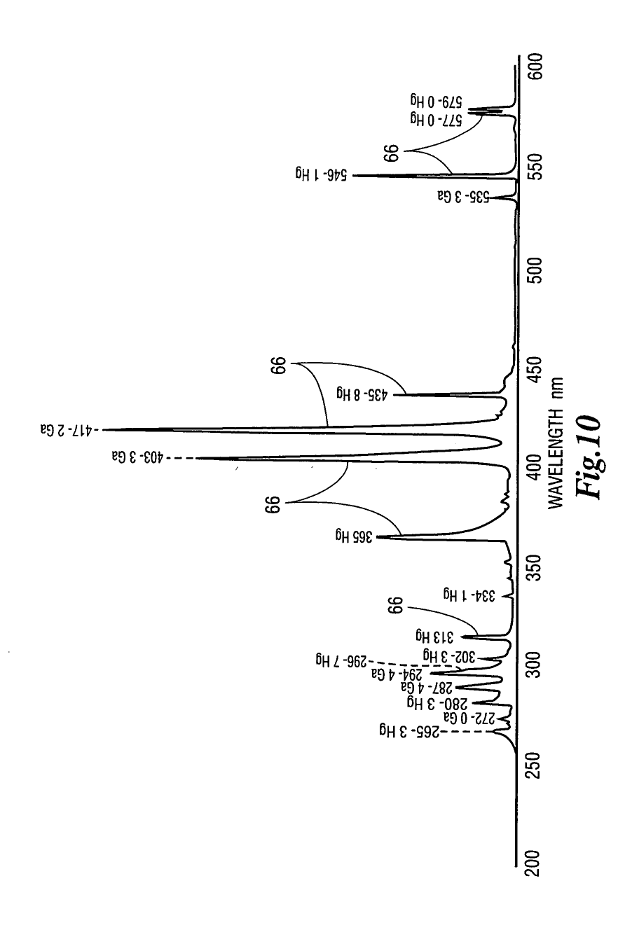

The graph of FIG. 9 containing plot 64 is prepared from the data of Table 1. The graph of FIG. 10 containing plot 66 is like that of FIG. 10 but indicates the chemicals in the lamp which give the spikes at the specific wavelengths.

Thus, to summarize, if the ultraviolet wavelengths (UV-A, UV-B, & UV-C) are combined in the proper proportions (approximately 35% UV-A, 40% UV-B, and 25% UV-C) and administered simultaneously to the micro-organism, in accordance with the method of the invention, the organism is reduced faster, more completely and without the complications (i.e. shadowing) attributed to traditional ultraviolet UV-C processing. Simply presented, when UV-A, UV-B and UV-C are (1) simultaneously and (2) in the correct proportionality administered to a micro-organism, the organism's cellular membranes are destroyed by the formation of “free radicals”, leaving the cell's DNA strand exposed and extremely vulnerable to UV-C. By this process, the (1) protective and (2) regenerative mechanisms of the cells of the micro-organism are destroyed simultaneously causing the cells and any cellular functions to immediately cease. The presence of rapid heat transfer, introduced to this process by infrared wavelengths (above 4000 A) from the UV lamp, increases the described, cellular destruction process.

The apparatus of the invention plays a critical role in the foregoing operation. First, the lamp 10 must be able to administer the stated proportionality of UV-A, UV-B and UV-C simultaneously. Second, the vessel 14 which the liquid passes through must maintain the liquid's contact with the lamp's ultraviolet rays for a time dependent on the lamp's intensity and the fluid's velocity through the vessel. The mercury gallium metal halide lamp 10 delivers the required proportionalities of UV-A, UV-B, and UV-C ultraviolet plus infrared light. The vessel 14 has an internal thread 23 extending the length of the vessel and protruding from the inner wall. This internal thread increases the distance the fluid must travel between the inlet and the outlet of the vessel. Maintaining a constant fluid velocity, the increased distance dictated by the vessel's internal thread causes an increase in the dwell time between the liquid and lamp. Furthermore, by increasing the internal thread's leads (i.e. double thread, triple thread etc.) and maintaining the same pitch, the required dwell time between liquid and lamp can be reduced due to the turbulence of the liquid caused by the multiple threads.

Successful animal and human clinical studies involving implanted devices have demonstrated no adverse reactions following synergistic inactivation and disinfection processing and complete compatibility with living cells. Dynamic sterilization and disinfection or synergistic isogenous activated decontamination (SIAD) has been examined with respect to the food processing industry. The process of the invention was found complimentary to both liquids and solids in microorganism reduction without altering product chemistry or physical composition.

The invention is illustrated further by the following comparison of the bactericidal effect of a photonic lamp 10 of the invention with a conventional ultraviolet lamp on a strain of Escherichia coli. The term “photonic lamp” refers to the multi-band or multi-spectrum UV lamp of the invention, as differentiated from conventional germicidal UV lamps operating only in the UV-C range. In particular a comparison was made of the bactericidal effect of a photonic lamp 10 of the invention with an ultraviolet (UV) light source at various distances from bacterial cell suspensions of Escherichia coli (E. coli) at varying sample depths.

The conventional ultraviolet lamp was one commercially available from Water Purification, Inc. under the designation Ultraviolet Lamp #FBO1 and having the following specifications: range: UVC; watts: 22; voltage: 117AC; amps: 0.19; and physical dimensions: 20 inch lighted length×0.625 inch diameter.

The lamp of the invention was an 8″ photonic lamp having the following specifications: range: UVA, UVB, and UVC; watts: 1500; voltage: 117AC; amps: 13; and physical dimensions: 8 inch lighted length×0.375 inch diameter.

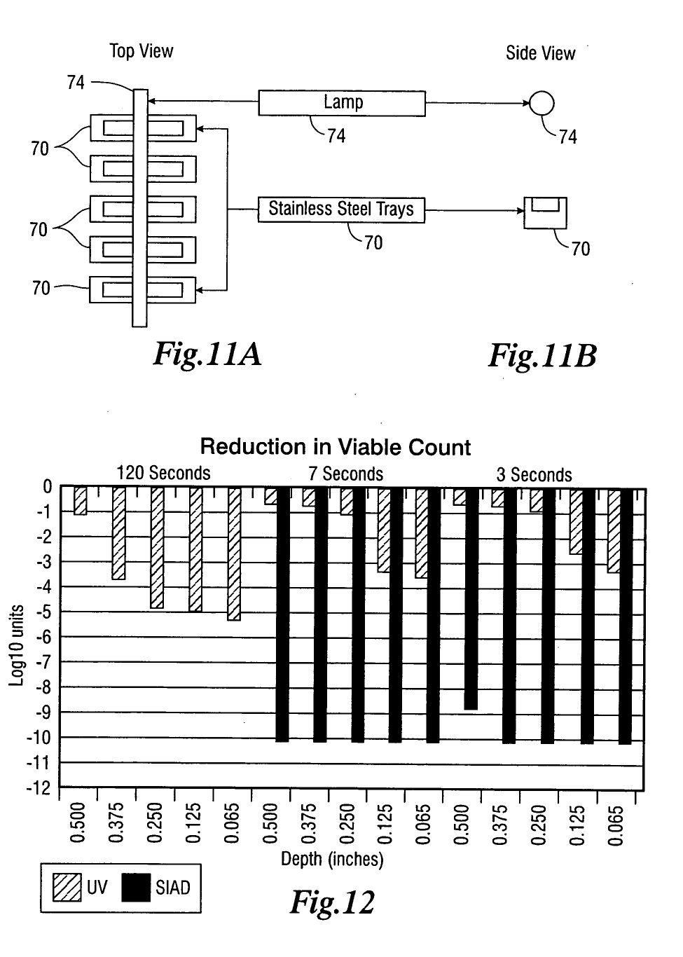

Escherichia coli is an enteric, gram-negative, motile, non-spore forming rod commonly used as an indicator organism for fecal contamination of water supplies. In this study a strain of E. coli (ATCC #47056) was grown on the surface of 10 Tryptic Soy Agar (TSA) plates and harvested after 18 hours of incubation at 37° C. in 5.0% CO₂ in air. The cells were suspended in sterile full-strength “Ringers” solution and placed in stainless steel trays 70 shown in FIG. 11A of varying capacities and depths as indicated by the entries in Tables 2 and 3. Saline was used rather than water to diminish the effect of cell lyses due to osmotic pressure. The trays were then placed directly below each lamp as shown in FIGS. 11A and 11B and exposed for periods of 3 and 7 seconds at a distance (R) of 2.25 inches from the lamp. In FIGS. 11A and 11B the reference numeral 74 represents both the conventional lamp and the lamp of the invention, the former being positioned in the arrangement of FIGS. 11A and 11B for the trials documented in Table 3 and the latter being positioned in that arrangement for the trials documented in Table 2. A third trial was conducted with the UV light using an exposure of 120 seconds. All trials included five varying depths (B) of cell suspension as indicated in Tables 2 and 3. Voltage, lamp temperature, and fluid temperatures at both the start and end of exposure were recorded at each trial for the photonic lamp of the invention throughout the study.

Following exposure, an aliquot of cell suspension was removed from each tray, plated onto TSA and incubated at 37° C., in air, for 24 hours. The recoverable cell counts were determined by plating appropriate dilutions onto TSA and compared to those of unexposed cell suspensions of E. coli. The results are summarized in Tables 2 and 3.

TABLE 2
Effect of Photonic Lamp of the Invention on 18 hr Culture of E. coli

| | | | | | | | | | Viable |
|---|---|---|---|---|---|---|---|---|---|
| | | Exposure | R¹ | B² | | Lamp | Fluid Temp | | Count |
| Trial# | Sample# | (sec.) | (in.) | (in.) | Voltage | Temp. | Start | End | CFU/ml |
| | 0 | Unexposed | N/A | N/A | N/A | N/A | N/A | N/A | 9.47E+09 |
| 1 | 1 | 7 | 2.25 | 0.500 | 523 | 150° C. | 21° C. | 22° C. | 0.00E+00 |
| | 2 | 7 | 2.25 | 0.375 | 523 | 150° C. | 21° C. | 22° C. | 0.00E+00 |
| | 3 | 7 | 2.25 | 0.250 | 523 | 150° C. | 21° C. | 22° C. | 0.00E+00 |
| | 4 | 7 | 2.25 | 0.125 | 523 | 150° C. | 21° C. | 22° C. | 0.00E+00 |
| | 5 | 7 | 2.25 | 0.065 | 523 | 150° C. | 21° C. | 22° C. | 0.00E+00 |
| 2 | 6 | 3 | 2.25 | 0.500 | 525 | 160° C. | 21° C. | 21° C. | 2.03E+01 |
| | 7 | 3 | 2.25 | 0.375 | 525 | 160° C. | 21° C. | 21° C. | 0.00E+00 |
| | 8 | 3 | 2.25 | 0.250 | 525 | 160° C. | 21° C. | 21° C. | 0.00E+00 |
| | 9 | 3 | 2.25 | 0.125 | 525 | 160° C. | 21° C. | 21° C. | 0.00E+00 |
| | 10 | 3 | 2.25 | 0.065 | 525 | 160° C. | 21° C. | 21° C. | 0.00E+00 |

TABLE 3
Effect of Ultraviolet Lamp #FB01 on 18 hr Culture of E. coli

| | | | | | | | | | Viable |
|---|---|---|---|---|---|---|---|---|---|
| | | Exposure | R¹ | B² | | Lamp | Fluid Temp | | Count |
| Trial# | Sample# | (sec.) | (in.) | (in.) | Voltage | Temp. | Start | End | CFU/ml |
| | 0 | Unexposed | N/A | N/A | N/A | N/A | N/A | N/A | 1.02E+10 |
| 1 | 1 | 120 | 2.25 | 0.500 | 117 | 25.7 | 25.0 | 25.0 | 1.06E+09 |
| | 2 | 120 | 2.25 | 0.375 | 117 | 25.7 | 25.0 | 25.0 | 2.85E+06 |
| | 3 | 120 | 2.25 | 0.250 | 117 | 25.7 | 25.0 | 25.0 | 1.77E+05 |
| | 4 | 120 | 2.25 | 0.125 | 117 | 25.7 | 25.0 | 25.0 | 1.48E+05 |
| | 5 | 120 | 2.25 | 0.065 | 117 | 25.7 | 25.0 | 25.0 | 7.32E+04 |
| 2 | 6 | 7 | 2.25 | 0.500 | 117 | 25.7 | 24.3 | 24.3 | 3.25E+09 |
| | 7 | 7 | 2.25 | 0.375 | 117 | 25.7 | 24.3 | 24.3 | 2.85E+09 |
| | 8 | 7 | 2.25 | 0.250 | 117 | 25.7 | 24.3 | 24.3 | 1.26E+09 |
| | 9 | 7 | 2.25 | 0.125 | 117 | 25.7 | 24.3 | 24.3 | 6.71E+06 |
| | 10 | 7 | 2.25 | 0.065 | 117 | 25.7 | 24.3 | 24.3 | 4.07E+06 |
| 3 | 11 | 3 | 2.25 | 0.500 | 117 | 25.7 | 24.5 | 24.5 | 3.41E+09 |
| | 12 | 3 | 2.25 | 0.375 | 117 | 25.7 | 24.5 | 24.5 | 2.68E+09 |
| | 13 | 3 | 2.25 | 0.250 | 117 | 25.7 | 24.5 | 24.5 | 1.79E+09 |
| | 14 | 3 | 2.25 | 0.125 | 117 | 25.7 | 24.5 | 24.5 | 3.90E+07 |
| | 15 | 3 | 2.25 | 0.065 | 117 | 25.7 | 24.5 | 24.5 | 7.11E+06 |

¹“R” = distance from the center of the lamp to the surface of the bacterial cell suspension.
²“B” = depth of sample.

Table 2 shows that the voltage and temperature of the photonic lamp of the invention remained relatively stable throughout the study. The voltage for trials 1 and 2 were 523 and 525 volts respectively while the lamp temperature ranged from 150° C. to 160° C. The fluid temperature remained stable and did not exceed 22° C., well within a range that would not alter viable cell counts.

Total viable count was reduced to undetectable levels in nine of ten samples exposed to the photonic lamp of the invention with an average cell recovery rate of 2.03 colony forming units (cfu)/ml (Table 2). This was in sharp contrast to the UV lamp exposures that resulted in an average cell recovery of 1.09×10⁹ cfu/ml (Table 3) representing an average drop in viable count of 2.35 log units compared to an average drop of almost 10 log units for the photonic lamp of the invention as further illustrated in the chart of FIG. 12. A decrease in bacterial kill was observed for the UV light samples with increased sample depth for all three time exposure times, while only one sample exposed to the photonic lamp of the invention.

The photonic lamp of the invention provided greater sample penetration and bactericidal effect than the ultraviolet light tested. This data suggests that the photonic lamp of the invention may be useful in the control and eradication of viable E. coli from water and other fluids.

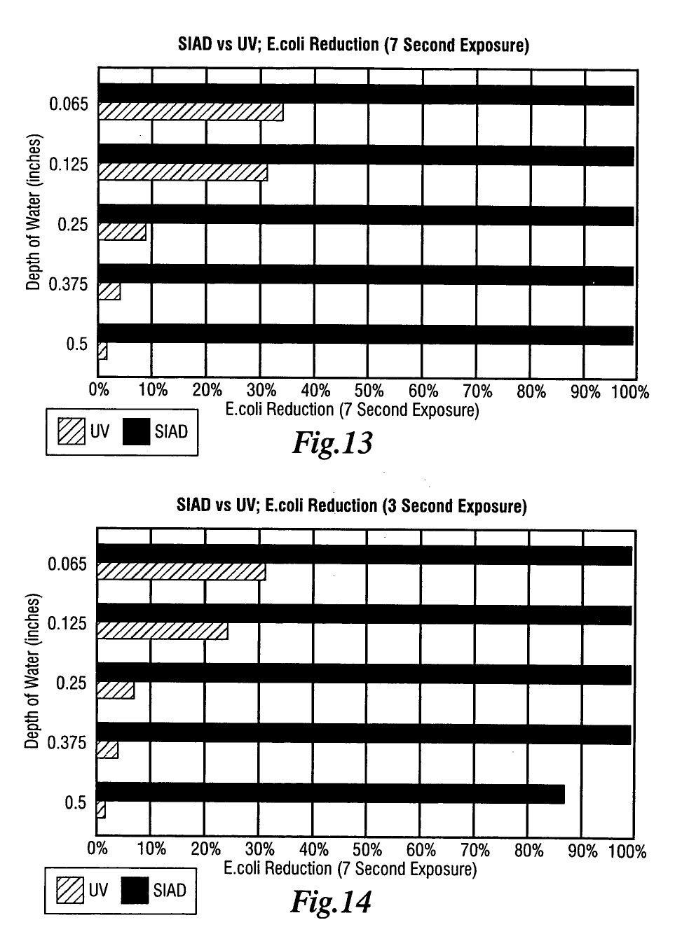

FIGS. 13 and 14 are charts comparing a water purification system utilizing the method and apparatus of the invention with a standard household UV-C water purification. In particular, FIG. 13 compares the two during a 7-second exposure, showing the extent of E. coli reduction and FIG. 14 compares the two during a 3 second exposure, showing the extent of E. coli reduction. By way of example, an illustrative water purification system utilizing the method and apparatus of the invention can be implemented using the arrangement of FIGS. 5A and 5B.

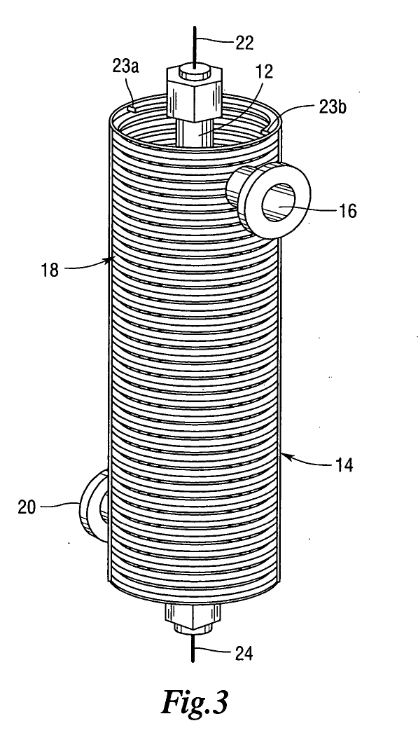

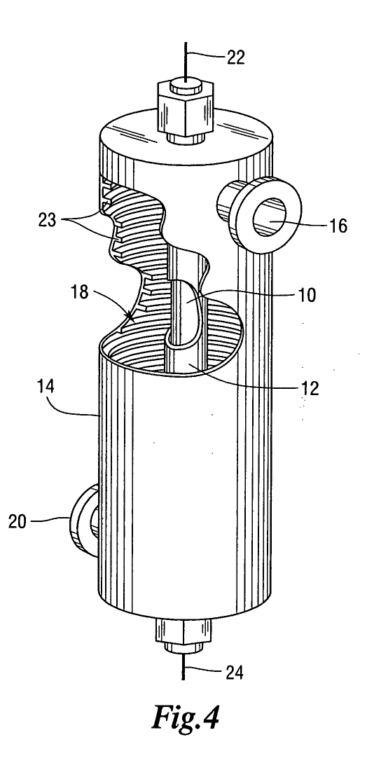

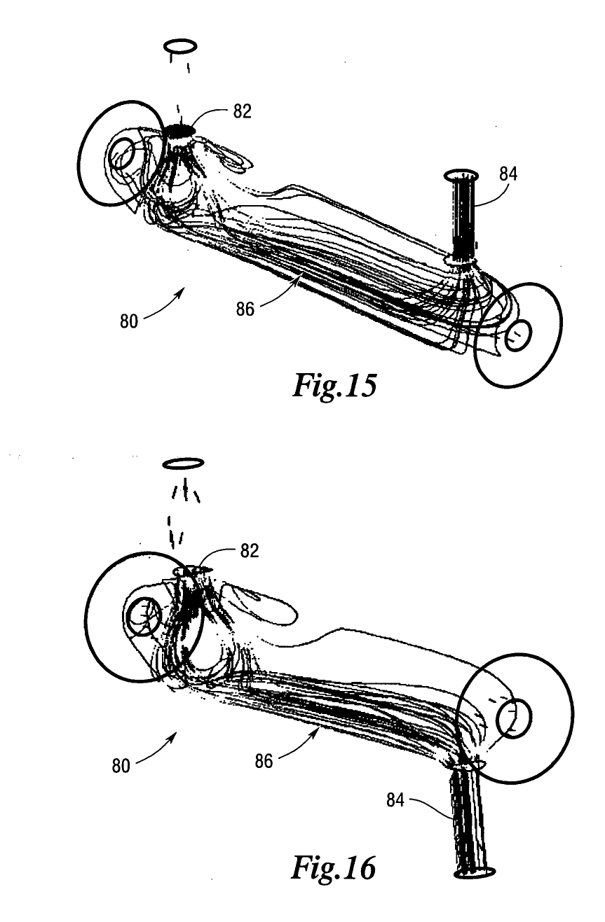

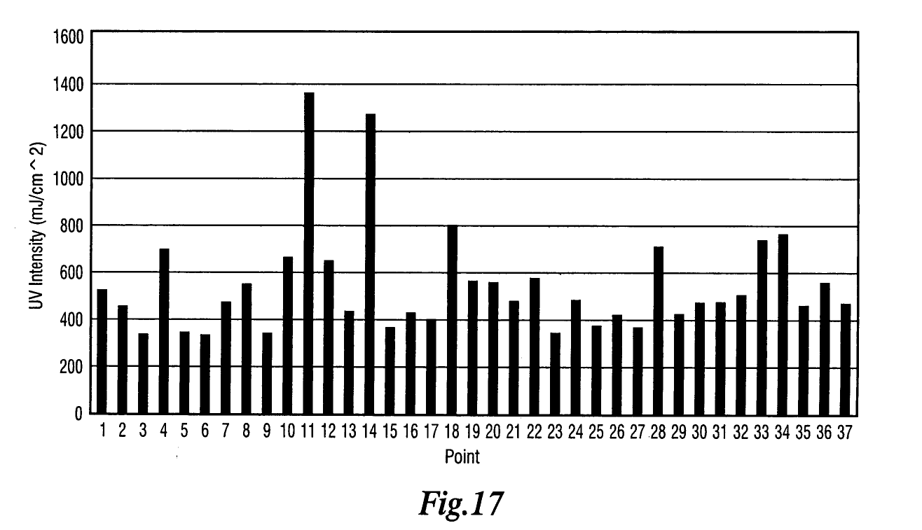

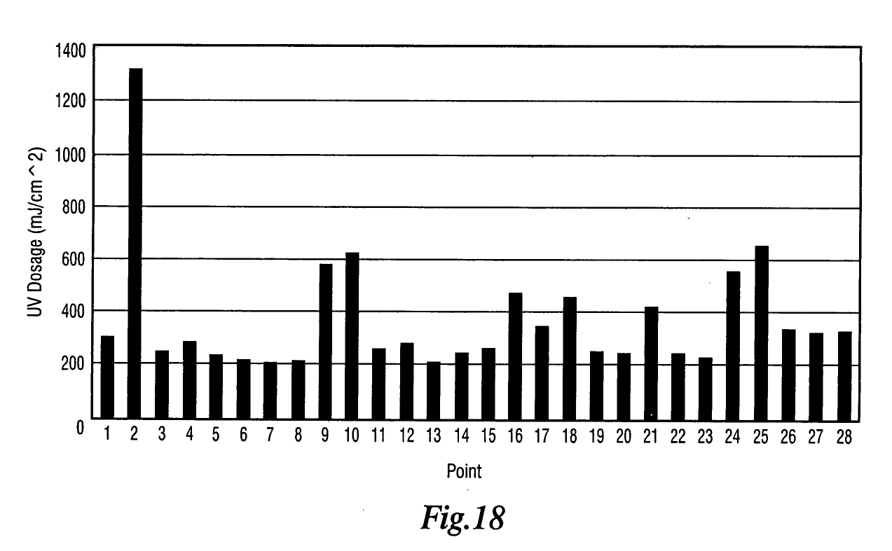

The method and apparatus of the invention illustrated in FIGS. 1-5 may be viewed as incorporating a flow processing mode of operation, in contrast to a relatively static, batch processing mode of operation. In addition, with the internal spiral geometry provided by threads 23 shown in FIGS. 3 and 4 and auger 45 shown in FIG. 5B, fluid particle irradiation advantageously is controlled and equalized rather than being random. In particular, FIG. 15 is a schematic diagram illustrating particle path fluid dynamics in a flow processing apparatus 80 similar to the apparatus shown in FIGS. 1-5 but without the internal spiral geometry, i.e. without threads 23 or auger 45. The apparatus 80 has an inlet 82 and outlet 84, and lines 86 represent the fluid particle flow paths from inlet 82 through the apparatus to the outlet. FIG. 16 is a schematic diagram like FIG. 15 wherein the components are identical and the outlet 84 is oriented in a direction opposite that shown in FIG. 15. FIGS. 17 and 18 are graphs illustrating particle irradiation in the arrangements of FIGS. 15 and 16, respectively, and it can be seen from these graphs that the fluid particle irradiation is random.

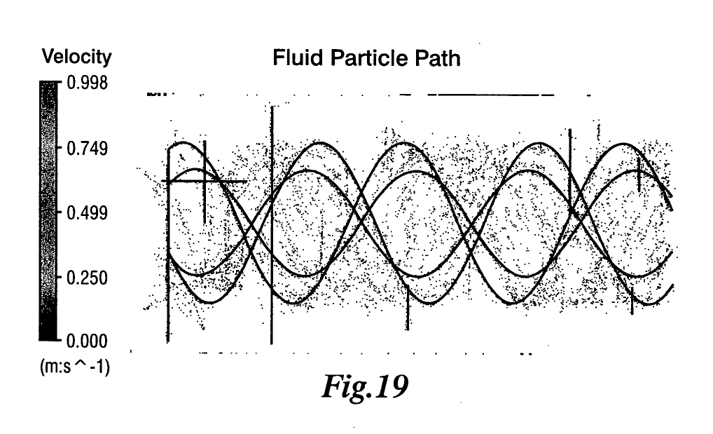

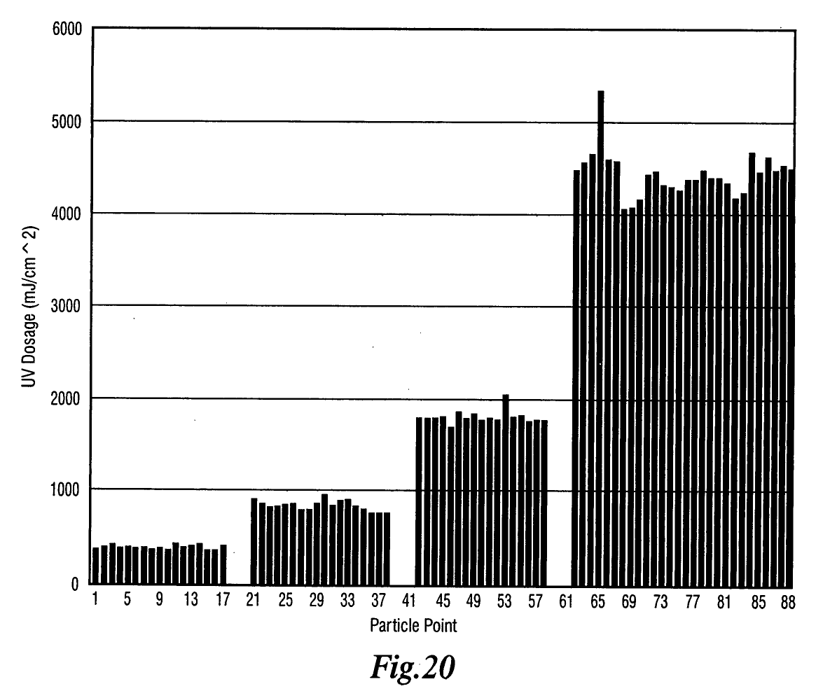

In arrangements incorporating the internal spiral geometry, illustrated for example in FIGS. 3, 4 and 5B, particle irradiation is controlled and equalized as compared to random irradiation associated with non-spiral arrangements. FIG. 19 is a fluid particle path velocity profile for the apparatus of the invention incorporating the internal spiral geometry, i.e. the threads 23 in the apparatus of FIGS. 1-4 and the auger 45 in the apparatus shown in FIG. 5B. FIG. 20 is a graph illustrating fluid particle irradiation in the apparatus of the invention having the internal spiral geometry. Each sector proceeding from left to right in FIG. 20 represents increased lamp wattage. For at least the first three sectors it can be seen that the fluid particle irradiation is controlled and equalized to be very uniform. Even the last sector, based on the highest lamp wattage, shows a generally uniform irradiation profile. Fluid particle irradiation being controlled and equalized advantageously results in efficient fluid particle irradiation and predictable increased fluid flow rates through the apparatus.

The invention is illustrated further by the following study of the bactericidal effect utilizing the invention as a nonthermal pasteurizer (SIAD TGIR) on bacterial contaminants in liquids. TGIR stands for Transportable Ground IRadiation. The purpose of this study was examine the effect of exposure time of the SIAD TGIR nonthermal pasteurizer on viable cell counts of previously reported bacterial contaminants in liquids.

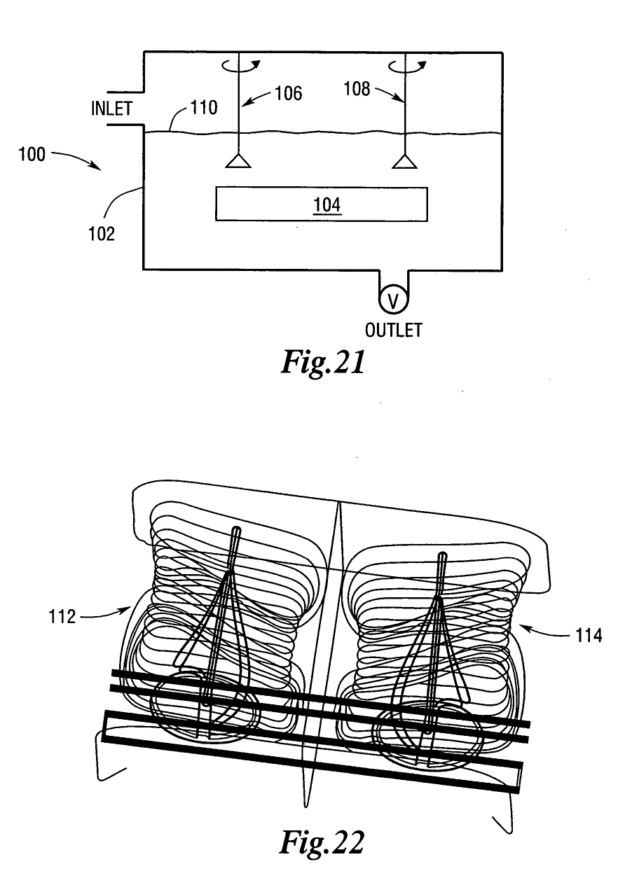

A non thermal pasteurizer according to the invention is illustrated in the schematic diagram of FIG. 21. Briefly, pasteurizer 100 includes a tank 102 into which a lamp 104 according to the invention is located. At least one motor driven mixing device is provided, in the present illustration two mixing devices 106, 108 are shown. Each device comprises a rod or shaft coupled at one end to a motor and provided at the end within tank 102 with a blade-like formation to provide a mixing action when the shaft is rotated. Liquid to be pasteurized is introduced to tank 102 as represented by the level designated 110 and remains therein for the predetermined pasteurization time. Lamp 104 is similar to lamp 10 shown in FIGS. 1-4 but does not have the threads 23. In this embodiment, which may be viewed as batch processing, the liquid flows around the exterior of lamp 104, the flow being a swirling type of flow promoted by the action of mixers 106, 1068. The mixers 106, 108 cause the fluid to go around and around lamp 104, and after a set time the fluid is drained from pasteurizer 100. Lamp 104 can be operated in the multi-band mode previously described, i.e. UV-A, UV-B and UV-C and in the output ratios about 35% UV-A, about 40% UV-B and about 25% UV-C.

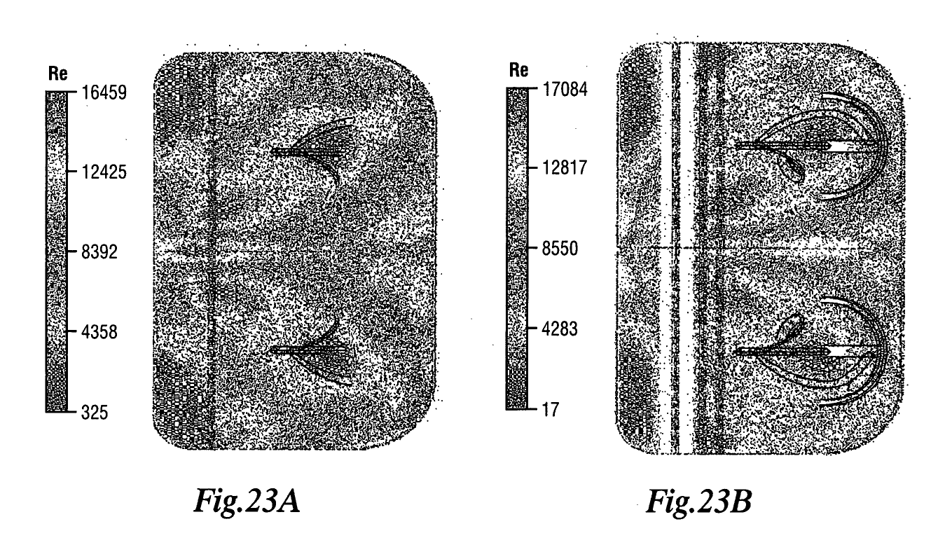

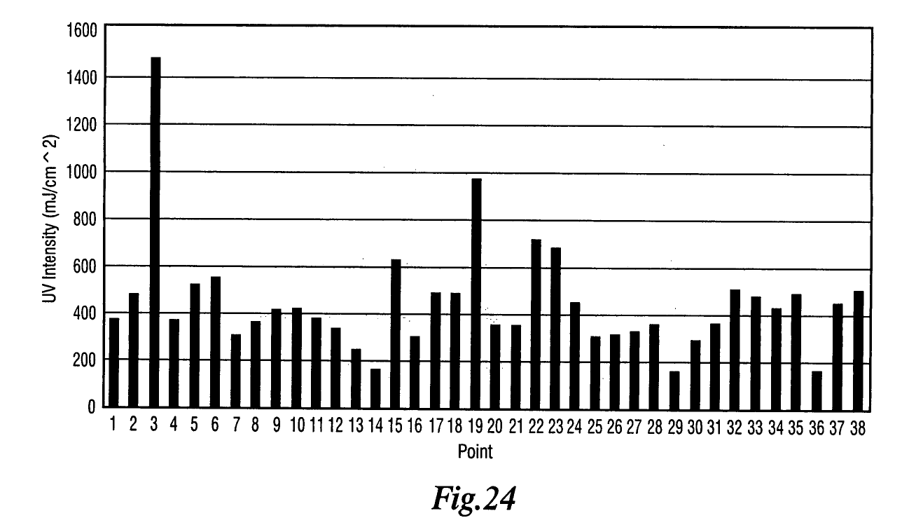

FIG. 22 is a schematic diagram illustrating particle path fluid dynamics in pasteurizer 100. Lines 112 and 114 represent the fluid particle flow paths associated with the two mixers. FIGS. 23A and 24B are graphs providing Reynolds Number plots associated with the illustration of FIG. 22. FIG. 24 is a graph showing particle irradiation in the pasteurizer illustrated in FIGS. 21 and 22 in the batch processing mode.

Included in the study were Salmonella choleraesuis subsp. choleraesuis serotype enteritidis ATCC strain 13076, Escherichia coli ATCC strain #43895 (O157:H7) and ATCC strain #47056, all acid tolerant, gram-negative, non-spore forming enteric rods. E. coli strain #47056 was used in some cases as a surrogate for ATCC #43895 the more pathogenic strain of E. coli. In addition, Bacillus cereus (ATCC #11778), a faculative, gram positive, spore forming rod, often used as a surrogate for Bacillus anthracis, was included.

Test strains maintained on the surface of Tryptic Soy Agar (TSA) were used to inoculate two to three liters of ½-strength Brain Heart Infusion broth. After 24 hours of incubation at 37° C. in air, the cells were harvested ascetically by centrifugation (15000 rpm for 20 min.), resuspended in sterile ¼ strength Ringer's solution, and used to inoculate seven liters of water, cider, and orange juice. After the SIAD TGIR nonthermal pasteurizer lamp reached a voltage of approximately 525 volts (−1 minute) aliquots were ascetically removed at predetermined times as indicated in Tables 4-9, diluted to concentrations of 10⁻², 10⁻⁴ and 10⁻⁴, then plated onto TSA using a Spiral Plater (Spiral Systems Inc.) and incubated at 37° C. in air. Maximum voltage and liquid temperature were recorded at each sample time and the speed of the mixer was maintained at 530 rpm. After 24 hours, recoverable cell count was determined and compared to that of an equivalent, unexposed cell suspension of each strain.

Water

Ringer's tablets were added to sterile distilled water to minimize cell lysis of the test strains due to the increased osmotic from the water. Enough tablets were added to give a final concentration ¼^{th} that of physiological saline.

Test strains: Escherichia coli (ATCC #47056), Bacillus cereus (ATCC #11778).

Orange Juice

Freshly squeezed as well as juice with heavy pulp content (Tropicana) were used in the study.

Test strains: Escherichia coli (ATCC #47056), Salmonella choleraesuis subsp. choleraesius serotype enteritidis (ATCC #13076).

Apple Cider

Fresh squeezed.

Test strain: Escherichia coli [ATCC #43895 (O157:H7)]

The test results for water are set forth in Tables 4 and 5.

Water

TABLE 4
Effect of SIAD TGIR Nonthermal Pasteurizer
with constant stirring on 24 hr
Culture of Escherichia coli (#47056)
in ¼ strength Ringer's solution

| | | Max. Temp | | |
|---|---|---|---|---|
| | | In Degrees | | Reduction in |
| Exposure (min.) | Volts | Centigrade | Cfu's/ml | Log Units |
| 0 | N/A | 26.5 | 8.33E+08 | N/A |
| 2 | 530 | 30.2 | 2.03E+01 | 7.61 |
| 4 | 529 | 32.1 | 0.00E+00 | 8.92 |
| 6 | 529 | 34.9 | 0.00E+00 | 8.92 |
| 8 | 529 | 37.5 | 0.00E+00 | 8.92 |
| 10 | 529 | 39.9 | 0.00E+00 | 8.92 |
| 15 | 529 | 48.9 | 0.00E+00 | 8.92 |

TABLE 5
Effect of SIAD TGIR Nonthermal Pasteurizer
with constant stirring on 24 hr
Culture of Bacillus cereus (ATCC #11778)
in ¼ strength Ringer's solution

| | | Max. Temp | | |
|---|---|---|---|---|
| | | In Degrees | | Reduction in |
| Exposure (min.) | Volts | Centigrade | ML/ml | Log Units |
| 0 | N/A | 23 | 8.33E+08 | N/A |
| 2 | 525 | 25 | 1.63E+08 | 0.71 |
| 4 | 525 | 29 | 0.00E+00 | 8.92 |
| 6 | 530 | 30 | 0.00E+00 | 8.92 |
| 8 | 531 | 33 | 0.00E+00 | 8.92 |
| 10 | 533 | 35 | 0.00E+00 | 8.92 |
| 30 | 534 | 51 | 0.00E+00 | 8.92 |

The test results for orange juice are set forth in Tables 6-8.

Orange Juice

TABLE 6
Effect of SIAD TGIR Nonthermal Pasteurizer
with constant stirring on 24 hr
Culture of Escherichia coli (#47056)
in orange juice with heavy pulp

| | | Max. Temp | | |
|---|---|---|---|---|
| | | In Degrees | | Reduction in |
| Exposure (min.) | Volts | Centigrade | ML/ml | Log Units |
| 0 | N/A | 7 | 4.39E+07 | N/A |
| 20 | 529 | 25 | 8.13E+03 | 3.73 |
| 30 | 529 | 33 | 1.22E+02 | 5.56 |
| 40 | 531 | 41 | 0.00E+00 | 7.64 |
| 50 | 529 | 48 | 0.00E+00 | 7.64 |
| 60 | 529 | 55 | 0.00E+00 | 7.64 |

TABLE 7
Effect of SIAD TGIR Nonthermal Pasteurizer
with constant stirring on 24 hr
Culture of Salmonella choleraesuis subsp.
choleraesuis serotype enteritidis (ATCC #13076)
in orange juice with heavy pulp

| | | Max. Temp | | |
|---|---|---|---|---|
| | | In Degrees | | Reduction in |
| Exposure (min.) | Volts | Centigrade | ML/ml | Log Units |
| 0 | N/A | 8 | 1.20E+07 | N/A |
| 10 | 527 | 19 | 2.03E+05 | 1.77 |
| 20 | 529 | 28 | 1.63E+04 | 2.87 |
| 30 | 530 | 35 | 1.34E+03 | 3.95 |
| 40 | 530 | 43 | 2.64E+02 | 4.66 |
| 50 | 530 | 52 | 0.00E+00 | 7.08 |
| 60 | 528 | 60 | 0.00E+00 | 7.08 |

TABLE 8
Effect of SIAD TGIR Nonthermal Pasteurizer
with constant stirring and bubbling carbon
dioxide gas on 24 hr Culture of
Salmonella cholersesuis subsp. choleraesuis
serotype enteritidis (ATCC #13076)
fresh squeezed orange juice

| | | Max. Temp | | |
|---|---|---|---|---|
| | | In Degrees | | Reduction in |
| Exposure (min.) | Volts | Centigrade | ML/ml | Log Units |
| 0 | N/A | 2 | 3.66E+06 | N/A |
| 5 | 529 | 10 | 1.02E+06 | 0.56 |
| 10 | 530 | 16 | 4.27E+04 | 1.93 |
| 15 | 529 | 20 | 1.22E+04 | 2.48 |
| 20 | 529 | 24 | 1.04E+03 | 3.55 |
| 25 | 527 | 29 | 2.44E+02 | 4.18 |
| 30 | 527 | 33 | 4.07E+01 | 4.95 |
| 40 | 525 | 43 | 0.00E+00 | 6.56 |
| 50 | 524 | 51 | 0.00E+00 | 6.56 |
| 60 | 525 | 60 | 0.00E+00 | 6.56 |

The test results for apple cider are set forth in Table 9.

Apple Cider

TABLE 9
Effect of SIAD TGIR Nonthermal Pasteurizer
with constant stirring on 24 hr
Culture of Escherichia coli (#43895) (O157:H7)
in Apple Cider

| | | Max. Temp | | |
|---|---|---|---|---|
| | | In Degrees | | Reduction in |
| Exposure (min.) | Volts | Centigrade | ML/ml | Log Units |
| 0 | N/A | 7 | 1.82E+08 | N/A |
| 15 | 545 | 22 | 2.03E+01 | 6.95 |
| 20 | 546 | 29 | 4.27E+02 | 5.63 |
| 30 | 547 | 37 | 1.83E+02 | 6.00 |
| 40 | 544 | 46 | 2.03E+01 | 6.95 |
| 50 | 545 | 55 | 0.00E+00 | 8.26 |
| 60 | 544 | 64 | 0.00E+00 | 8.26 |

Escherichia coli (Table 4) and Salmonella choleraesuis subsp. choleraesuis serotype enteritidis (Table 5) levels in water (¼ strength Ringer's solution) were reduced by more than 5 log units after two minutes of exposure to the SIAD TGIR Nonthermal Pasteurizer and viable cells could not be recovered after four minutes. Escherichia coli (Table 6) and Salmonella choleraesuis subsp. choleraesuis serotype enteritidis (Table 7) levels in orange juice with heavy pulp (¼ strength Ringer's solution) were reduced 5.56 and 3.95 Log units respectfully after thirty minutes of exposure to the SIAD TGIR Nonthermal Pasteurizer and viable cells could not be recovered after forty and fifty minutes, respectively. Carbon dioxide gas was bubble through fresh squeezed orange juice to preserve nutritional content and to improve the taste of the juice. The data in Table 8 demonstrates that the addition of CO₂ to the process had little if any change in bactericidal effect.

The level of the pathogenic strain of Escherichia coli (ATCC #43895) in apple cider was reduced by more than 5 Log units after 15 minutes of exposure and was not recoverable after fifty minutes of exposure.

In conclusion the SIAD TGIR nonthermal pasteurizer process of the invention achieved a 5 Log reduction in viable cell count with Escherichia coli (ATCC #47056) in orange juice and water, with Escherichia coli [ATCC #43895 (O157:H7)] in apple cider, with Salmonella choleraesuis subsp. choleraesuis serotype enteritidis (ATCC #13076) in orange juice, and with Bacillus cereus (ATCC #11778) in water and was able to reduce the level of all test organisms to undetectable levels over time.

By way of example, in an illustrative pasteurizer of the invention, using a 1500 watt lamp of the invention, FDA 5 log reduction was achieved in about 25 minutes on 6 gallons of orange juice and in about 2 minutes on 6 gallons of water. No nutritional changes in either the orange juice or the water were observed. Using a 4 inch, 400 watt lamp of the invention, FDA 5 log reduction was achieved in about 20 seconds on 3 quarts of water. Again, no nutritional changes were observed.

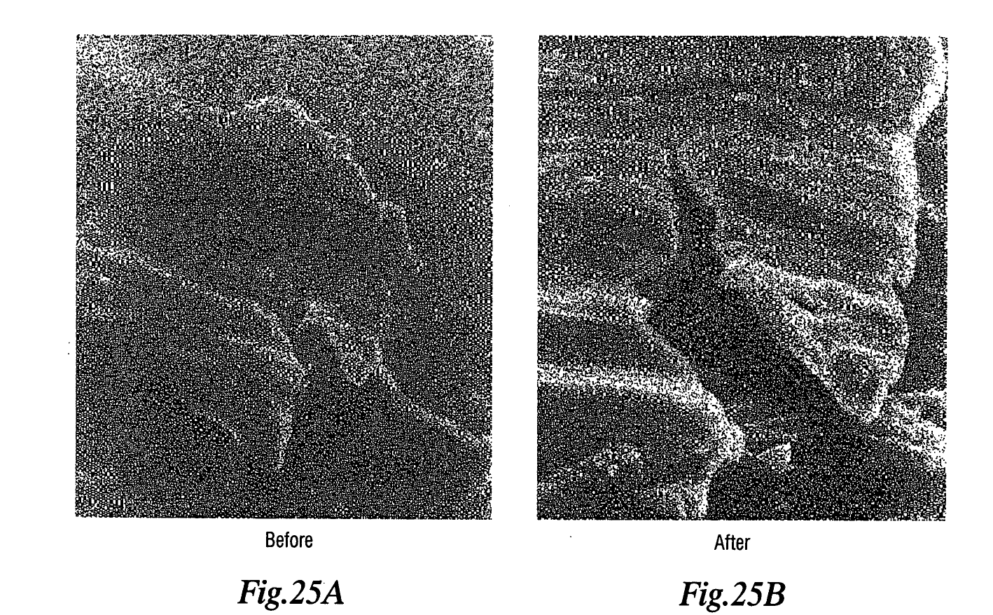

FIGS. 25A and 25B are scanning electron microscope photographs further illustrating the invention. In particular, they show the effect of a 10-second treatment of bacillus subtilis spores using the process of the invention. The spherical shaped spores are seen in FIG. 25A before application of the process of the invention. The process of the invention, utilizing UV-A, UV-B and UV-C for a duration of 10 seconds, completely destroyed the spores, reducing the microorganism to deposits of carbon, as evidenced by the absence of the spores in the photograph of FIG. 25B.

The invention is illustrated further by a nutrient analysis of orange juice and apple cider before and after treatment by the method of the invention. Many nutrients can be affected by irradiation and temperature. Both of these might be important components of the system and method of the invention. After the conditions needed for biological decontamination of the juices being studied are determined, samples that have been treated by the system and method of the invention will be analyzed for nutrient content. The nutrients that will be measured are those with potential to be damaged by the high intensity photonics and rapid heat transfer. In addition, only those nutrients that are present in significant levels in orange juice will be studied. The vitamins of interest in orange juice (per 8 fl oz, 116 kcal) for this project include Vitamin C (125 mg, 200% DRI), Thiamin (0.2 mg, 15% DRI), Riboflavin (0.074 mg, 5% DRI), and Folate (75 ug, 20% DRI).

Vitamin C (L-ascorbic acid and dehydro-L-ascorbic acid) is an essential nutrient, first related to the ancient disease scurvy. It is reported to be the basis of the first clinical trial on the lack of food component and disease by James Lind in 1753. It functions in the human as an antioxidant and as an enzyme cofactor for such reactions as collagen synthesis, carnitine and norepinephrine synthesis and liver detoxification. The current DRI for Vitamin C ranges from 15 mg/d for children aged 1 to 3 years to 120 mg/d for lactating women with an additional 35 mg/d for smokers. Degradation of L-ascorbic acid occurs in aqueous solutions in several conditions; low pH, high temperature, presence of oxygen and metals (copper as an example). Orange juice is an excellent media for preserving vitamin C if temperature is kept low and light is excluded, since the pH is low. Fresh orange juice is also an excellent source due to the lack of heat processing. The exposure to light in the process of the invention is an important issue. The first degradation reaction of L-ascorbic acid is to dhydroascorbic acid (DHAA). DHAA may then be irreversibly converted to 2,3 keto-L-gluonic acid.

Thiamin was one of the first of the water soluble vitamins to be identified, being an important cofactor for many enzymes (several involving energy metabolism) and also having some non-coenzyme functions. Diseases related to the deficiency of thiamin have been known for thousands of years, in particular beriberi. Oranges can be a significant source of thiamin, there being almost as much thiamin in a single medium orange as in a slice of enriched white bread. Thiamin is unstable in the presence of oxygen and heat; however its stability is better when pH is less than 7 as in orange juice. So a low temperature process in the absence of oxygen could be the best situation to preserve thiamin in aqueous solutions such as orange juice.

Riboflavin, as known as B₂, is an integral part of two key coenzymes (FAD and FMN). As FAD and FMN, riboflavin is crucial for energy metabolism, drug metabolism, and antioxidant functions. Riboflavin is stable to heat sterilization, but is very sensitive to light and oxidation. Light therapy for some newborns is known to cause riboflavin deficiency. The sensitivity to light has been a major reason for discontinuing the use of glass milk bottles with up to half the riboflavin being lost with 2 hours of exposure to bright sunlight. This sensitivity of light is related to production of reactive free radicals which react with riboflavin. As with thiamin, the level in a single orange is not much different than a slice of enriched white bread.

Folic acid or folate occurs in many different forms making it difficult to measure accurately, such as 5-methyl-FH₄ and 10 formyl-FH₄. In addition, folate also has various degrees of polyglutamine. Orange juice has a mix of folate glutamate, mono, and penta being the most common. This makes it imperative to measure folate by a bioassay. Folate is critical for nucleic acid and protein metabolism with several well-known deficiency diseases, such as megaloblastic anemia. Folate, of all of the other vitamins studied, is very liable to UV light, oxygen, and heat. It is most sensitive to light in the presence of oxygen. Oxidation leads to the conversion of FH₄ to dihydrofolic acid and fully oxidized folate. Eventually the process of oxidation leads to inactive forms. This is especially true in acidic media where the inactive 5-methyl-5,8-FH₂. Therefore it may be essential to avoid the presence of oxygen to preserve the active folic in foods.

Fresh orange juice can be an important source of three of these vitamins: C, Thiamin, and Folate. The fourth vitamin, riboflavin, even though not present at significant dietary levels, was studied due to its sensitivity to oxidation. The results could be useful in determining the effect of the system and method of the invention on other nutrients and oxidation in general. Therefore it was decided that it was necessary that these vitamins be assayed before and after treatment using the system and method of the invention.

A vitamin analysis was first carried out on apple juice and orange juice without CO₂ being present. Folate, thiamin, riboflavin, and vitamin C were analyzed in both Orange and Apple Juice. Vitamin C was measured by a fluorometric assay that measures both the oxidized and reduced forms of Ascorbic acid. Reference may be made to Deutsch, M. J. (Assoc. Ch. ed., 1990) “Vitamins and other Nutrients,” Chapter 45 in Hlerich, K. (ed.) Official Methods of Analysis of the Association of Official Analytical Chemists. 15^{th} Edition Volume 2. Association of the Official Analytical Chemists, Inc. Arlington, Va., Method No. 967.22 “Vitamin C (Ascorbic Acid) in Vitamin Preparations: Microfluorometric Method,” 1059-1060. [AOAC 967.22]

In brief, samples are homogenized then extracted with 4% (w/v) Metaphosphoric acid—MeOH pH 2.1. After being filtered through activated charcoal, a subsample is treated with boric acid-sodium acetate to prepare a blank while another portion is treated with sodium acetate. These samples are reacted with o-phenylenediamine to produce a fluorophor. After standing for 35 min. (protected from light) fluorescence is measured. Thiamine was measured by the flurometric assay [AOAC 942.23]. Reference may be made to Deutsch, M. J. (Assoc. Ch. ed., 1990). “Vitamins and Other Nutrients,” Chapter 45 in Hehich, K. (ed) Official Methods of Analysis of the Association of Official Analytical Chemists, 15^{th} Edition Volume 2. Association of Official Analytical Chemists, Inc. Arlington, Va., Method No. 942.23 “Thiamine in Foods: Fluorometric Method.” Samples are homogenated before analysis. In brief, NaCl or KCl was mixed with the standard or sample solution into a reaction vessel. Alkaline KFe (CN) is then added and gently swirling by a rotary motion. Then isobutanol is added to the reaction vessel, which is stoppered and shaken vigorously followed by centrifugation at low speed. The lower phase is removed and anhydrous sodium sulfate is added to the isobutanol layer with vortexing and shaking. The fluorescence of isobutanol extract is measured using a fluorometer. Riboflavin content was determined by a microbiological assay. Reference may be made to Baker, H., and Frank O. in Riboflavin, Rivlin, R. S. (ed) Plenum Press, NY: p 49.

Samples are autoclaved with 0.1 HCl to liberate the flavins. Samples are then filtered. The growth of Lactobacillus casei is used as the endpoint. Folate was analyzed by a microbiological assay using Lactobacillus casei. Reference may be made to Wight, A. J. A. and Phillips, D. R. Brit. J. Nutr., 53:569 (1985).

Juice samples were Tropicana Pure Premium Orange Juice (not from concentrate) with lots of pulp and Tops brand Walter 100% Apple Juice. The juice samples were refrigerated (5C) before treatment by the system and method of the invention. Samples were collected from the treatment at three time points: first after mixing, second after 20 minutes and finally after 40 minutes. After collection samples were chilled to 5C before being frozen at −20C.

Assayed folate increased during the process in Apple juice and during the first 20 minutes in the Orange Juice but decreased to below detectable limits in the final sample at 40 minutes. These results could be due to an increased solubility of this nutrient. The eventual loss of folate after 40 minutes may be the result of either the presence of oxygen or the increased temperature or both. Samples increased in temperature during the process from around 12C to 50C by 40 minutes.

Apple juice had no detectible riboflavin or vitamin C. There was a significant loss of Vitamin C and Riboflavin in the Orange Juice during the process. This is probably due to the oxygen and heat. The results are summarized in Table 10.

TABLE 10
Apple Juice

| | | 20 minutes | 40 minutes |
|---|---|---|---|
| Per 100 g | Before 11 C. | 27 C. | 45 C. |
| Folate, ug | 3.83 | 10.5 | 22.7 |
| Thiamine, ug | 10 | 30 | 10 |
| Riboflavin, ug | <20* | <20* | <20* |
| Vitamin C, mg | <1* | <1* | <1* |

Orange Juice

| | | 20 minutes | 40 minutes |
|---|---|---|---|
| Per 100 g | Before 13 C. | 30 C. | 49 C. |
| Folate, ug | 26.3 | 47.4 | <6* |
| Thiamine, ug | 60 | 60 | 40 |
| Riboflavin, ug | 30 | 20 | <20* |
| Vitamin C, mg | 27.9 | 12.0 | 1.1 |

*Values below detectable limits.

Next, a vitamin analysis was carried out on orange juice with co₂ being present. Juice samples were Jennings Citrus fresh squeezed—not pasteurized—orange juice. The juice samples were refrigerated (5C) before treatment by the system and method of the invention. Samples of Jennings Citrus orange juice were collected at 0, 25 and 45 minutes during treatment. Samples were refrigerated (5C) then frozen (−20C) before analysis. All the vitamins were analyzed by the same methodology except vitamin C which analyzed by a different procedure than done with the non-CO2 samples. Total ascorbic acid was determined by the dinitrophenylhydrazine method. Reference may be made to Burtis C. and Ashwood E. (eds); Dinitrophenyl hydrazine Method; Tietz Textbook of Clinical Chemistry 2^{nd} Edition; W.B. Saunders Co.; 1994; pp 1313-1314. 2,4-dinitrophenylhydrazine, ascorbic acid, thiourea and CuSO₄ were purchased from Sigma. Sulfuric and meta-phosphoric acid (MPA) were purchased from J. T. Baker (Phillipsburg, N.J.). Samples for analysis were stabilized by adding 0.5 mL juice sample to 2.0 ml 6% MPA and centrifuging at 3,000×g for 10, minutes. Clear supernatant was aspirated for batch analysis. Standards encompassing the range from 0-2.0 mg/dL ascorbic were prepared fresh for each analysis in 6% MPA. Color reagent was prepared prior to each analysis by mixing 100 mL dinitrophenylhydrazine (2.0 g/dL in 4.5 M sulfuric acid), 5.0 mL CuSO₄ (0.6 g/dL in H₂O) and 5.0 mL thiourea (5.0 g/dL in H₂O). For assay, MPA supernatants were diluted by 10-fold serial dilution and 0.6 mL of each dilution (1:1 to 1:10,000) or standard in MPA was mixed with 0.2 mL color reagent and incubated at 37° C. for 3 hours. Samples were chilled on ice for 10 minutes and then vortexed during addition of 1.0 mL 12M H₂SO₄. Care was taken to insure that the temperature of the sample did not exceed room temperature. Absorbance of each sample was determined at 520 nm on a shimadzu 160 UV spectrophotometer and plotted against concentration. The results are summarized in Table 11.

TABLE 11
Orange Juice with CO₂

| | Before | 20 minutes | 40 minutes |
|---|---|---|---|
| Per 100 mL | 13 C. | 31 C. | 45 C. |
| Folate, ug | 28.2 | 32.4 | 24.3 |
| Thiamine, ug | 70 | 80 | 70 |
| Riboflavin, ug | 30 | 30 | 20 |
| Vitamin C, mg | 48 | 48 | 50 |

None of the four vitamins that were measured showed a large drop in their content with the treatment by the system and method of the invention with CO₂.

From the foregoing it is apparent that the invention accomplishes its intended objectives. While embodiments of the invention have been described in detail, that has been done for the purpose of illustration, not limitation.

1. Apparatus for disinfection/pasteurization of fluids comprising:
a) a mercury/gallium metal halide ultraviolet lamp enclosed within an ozone free metallic doped quartz envelope;
b) an ozone free, metallic doped quartz enclosure for the lamp; and
c) a vessel containing the lamp in an enclosure and an in-line stationary spiral lead surrounding the enclosure and the vessel having an inlet, an outlet and a chamber in fluid communication therewith defining a flow path for fluid to be disinfected/pasteurized.

2. Apparatus according to claim 1, wherein the lamp is in the form of a tube and the enclosure and the vessel are generally cylindrical in shape, with the lamp, enclosure, and vessel being in generally concentric relation.

3. Apparatus according to claim 2, wherein the inlet and outlet are at opposite ends of the vessel.

4. Apparatus according to claim 2, wherein the diameter of the vessel is about twice the diameter of the enclosure, and wherein the diameter of the enclosure is about twice the diameter of the lamp.

5. Apparatus according to claim 1, wherein the lamp operates in a wavelength range from about 100 nanometers to about 400 nanometers and at a temperature ranging from about 600 degrees centigrade to about 800 degrees centigrade.

6. Apparatus according to claim 1, wherein the enclosure allows transmission of ultraviolet radiation from the lamp to the fluid without buildup of ozone.

7. Apparatus according to claim 1, wherein the spiral contains multiple leads.

8. Apparatus according to claim 1, wherein the lamp provides ultraviolet radiation having wavelength bands designated UV-A, UV-B and UV-C wherein band UV-A has a wavelength from about 315 nanometers to about 400 nanometers, band UV-B has a wavelength from about 280 nanometers to about 315 nanometers and band UV-C has a wavelength from about 100 nanometers to about 280 nanometers.

9. Apparatus according to claim 8, wherein the lamp provides output ratios of about 35% UV-A, 40% UV-B and 25% UV-C.

10. Apparatus according to claim 9, wherein the lamp provides the output ratios simultaneously.

11. Apparatus for disinfection/pasteurization of fluids comprising:
a) a mercury/gallium metal halide ultraviolet lamp enclosed within an ozone free metallic doped quartz envelope, the lamp providing ultraviolet radiation having wavelength bands designated UV-A, UV-B and UV-C wherein band UV-A has a wavelength from about 315 nanometers to about 400 nanometers, band UV-B has a wavelength from about 280 nanometers to about 315 nanometers and band UV-C has a wavelength from about 100 nanometers to about 280 nanometers;
b) an ozone free, metallic doped quartz enclosure for the lamp; and
c) a vessel containing the lamp and enclosure and having an inlet, an outlet and a chamber in fluid communication therewith defining a flow path for fluid to be disinfected/pasteurized.

12. Apparatus according to claim 11, wherein the lamp provides output ratios of about 35% UV-A, 40% UV-B and 25% UV-C.

13. Apparatus according to claim 12, wherein the lamp provides the output ratios simultaneously.

14. A method for disinfection/pasteurization of fluids comprising:
a) providing a mercury/gallium metal halide ultraviolet lamp enclosed within an ozone free metallic doped quartz envelope;
b) providing an ozone free, metallic doped quartz enclosure for the lamp;
c) providing a vessel containing the lamp, enclosure and an in-line stationary spiral lead surrounding the enclosure, the vessel having an inlet, an outlet and a chamber in fluid communication therewith defining a flow path for fluid to be disinfected/pasteurized; and
d) operating the lamp to introduce ultraviolet radiation and heat from the lamp into the fluid with the enclosure preventing build up of ozone.

15. The method according to claim 14, wherein the lamp is operated in a wavelength range from about 100 nanometers to about 400 nanometers.

16. The method according to claim 14, wherein the lamp is operated at a temperature ranging from about 600 degrees centigrade to about 800 degrees centigrade.

17. The method according to claim 14, wherein the fluid is a liquid.

18. The method according to claim 14, wherein the spiral contains multiple leads.

19. The method according to claim 14, wherein the lamp provides ultraviolet radiation having wavelength bands designated UV-A, UV-B and UV-C wherein band UV-A has a wavelength from about 315 nanometers to about 400 nanometers, band UV-B has a wavelength from about 280 nanometers to about 315 nanometers and band UV-C has a wavelength from about 100 nanometers to about 280 nanometers.

20. Apparatus according to claim 19, wherein the lamp provides output ratios of about 35% UV-A, 40% UV-B and 25% UV-C.

21. Apparatus according to claim 20, wherein the lamp provides the output ratios simultaneously.

22. A method for disinfection/pasteurization of fluids comprising:
a) providing a mercury/gallium metal halide ultraviolet lamp enclosed within an ozone free metallic doped quartz envelope;
b) providing an ozone free, metallic doped quartz enclosure for the lamp;
c) providing a vessel containing the lamp and enclosure and having an inlet, an outlet and a chamber in fluid communication therewith defining a flow path for fluid to be disinfected/pasteurized; and
d) operating the lamp to introduce ultraviolet radiation and heat from the lamp into the fluid with the enclosure preventing build up of ozone, the lamp being operated to provide ultraviolet radiation having wavelength bands designated UV-A, UV-B and UV-C wherein band UV-A has a wavelength from about 315 nanometers to about 400 nanometers, band UV-B has a wavelength from about 280 nanometers to about 315 nanometers and band UV-C has a wavelength from about 100 nanometers to about 280 nanometers.

23. The method according to claim 22, wherein the lamp provides output ratios of about 35% UV-A, 40% UV-B and 25% UV-C.

24. The method according to claim 23, wherein the lamp provides the output ratios simultaneously.

25. The method according to claim 22 further including providing an in-line stationary spiral lead surrounding the enclosure.

26. The method according to claim 25 further including multiple spiral leads surrounding the enclosure.

27. Fluid pasteurization apparatus comprising:
a) a tank having an inlet and an outlet;
b) a mercury/gallium metal halide ultraviolet lamp located within the tank, the lamp being enclosed within an ozone free, metallic doped quartz enclosure; and
c) at least one mixing device within the tank for causing fluid to be pasteurized to flow around the lamp.

28. Fluid pasteurization apparatus according to claim 27, wherein the lamp provides ultraviolet radiation having wavelength bands designated UV-A, UV-B and UV-C wherein band UV-A has a wavelength from about 315 nanometers to about 400 nanometers, band UV-B has a wavelength from about 280 nanometers to about 315 nanometers and band UV-C has a wavelength from about 100 nanometers to about 280 nanometers.

29. Fluid pasteurization apparatus according to claim 28, wherein the lamp provides output ratios of about 35% UV-A, 40% UV-B and 25% UV-C.

30. A fluid pasteurization method comprising:
a) providing a tank;
b) placing within the tank a mercury/gallium metal halide ultraviolet lamp being enclosed within an ozone free, metallic doped quartz enclosure;
c) introducing fluid to be pasteurized into the tank so that the lamp is immersed in the fluid; and
d) mixing the fluid to cause it to flow around the lamp while the lamp emits ultraviolet radiation.

31. The method according to claim 30, wherein the lamp provides ultraviolet radiation having wavelength bands designated UV-A, UV-B and UV-C wherein band UV-A has a wavelength from about 315 nanometers to about 400 nanometers, band UV-B has a wavelength from about 280 nanometers to about 315 nanometers and band UV-C has a wavelength from about 100 nanometers to about 280 nanometers.

32. The method according to claim 31, wherein the lamp provides output ratios of about 35% UV-A, 40% UV-B and 25% UV-C.
